(19) United States
(12) Patent Application Publication
(10) Pub. No.: US 2026/0051403 A1
(43) Pub. Date: Feb. 19, 2026

(54) LARGE LANGUAGE MODEL BASED PATIENT TO CLINICAL TREATMENT CRITERIA MATCHING WITH CONFIDENCE SCORES

(71) Applicant: GE Precision Healthcare LLC, Waukesha, WI (US)

(72) Inventors: Akshit Achara, London (GB); Sanand Sasidharan, Bangalore (IN)

(21) Appl. No.: 18/986,133

(22) Filed: Dec. 18, 2024

(30) Foreign Application Priority Data
Aug. 16, 2024 (IN) 202441062085

(51) Int. Cl. G16H 50/20; G16H 10/20

(52) U.S. Cl. CPC G16H 50/20; G16H 10/20

(57) ABSTRACT

Techniques for large language model (LLM) based patient to clinical treatment criteria matching with confidence score generation are described. In an example, a computer-implemented method can comprise generating different variations of textual input data for a LLM configured to generate an inference response to a clinical question regarding a patient and having a categorical answer, wherein the textual input data comprises a textual prompt of the clinical question, patient data comprising electronic medical record information for the patient and clinical criteria data comprising clinical criteria related to the clinical question. The method further comprising applying the LLM to the different variations and generating inference responses for each of the different variations, determining a final inference response to the clinical question based on a combination of the inference responses, and generating a confidence score for the final inference response based on a measure of variability between the inference responses.

### CROSS-REFERENCE TO RELATED APPLICATIONS

This application claims priority to India Provisional Patent Application No. 202441062085 filed on Aug. 16, 2024, entitled “LARGE LANGUAGE MODEL BASED PATIENT TO CLINICAL TREATMENT CRITERIA MATCHING WITH CONFIDENCE SCORES”. The entireties of the aforementioned application are incorporated by reference herein.

### TECHNICAL FIELD

This application relates to artificial intelligence (AI) in the medical domain and more particularly to large language model (LLM) based patient to clinical treatment criteria matching with confidence scores.

### BACKGROUND

Recommending a suitable clinical treatment for a patient, out of the thousands of potential options, is a time-consuming process for a doctor. The challenge is in comparing every aspect of patient's medical history with the inclusion and exclusion criteria of the clinical or different treatment options.

### SUMMARY

The following presents a summary to provide a basic understanding of one or more embodiments of the invention. This summary is not intended to identify key or critical elements or delineate any scope of the different embodiments or any scope of the claims. Its sole purpose is to present concepts in a simplified form as a prelude to the more detailed description that is presented later. In one or more embodiments, systems, computer-implemented methods, apparatus and/or computer program products are described that facilitate large language model (LLM) based patient to clinical treatment criteria matching with confidence scores.

According to an embodiment, a system is provided that comprises a memory that stores computer-executable components, and a processor that executes the computer-executable components stored in the memory. The computer-executable components can comprise an input variation component that generates different variations of textual input data for an artificial intelligence (AI) model (e.g., an LLM) configured to generate an inference response to a clinical question regarding a patient, wherein the textual input data comprises a textual prompt of the clinical question, patient data comprising electronic medical record information for the patient and clinical criteria data comprising clinical criteria related to the clinical question, and wherein the inference response comprises an answer value of amongst a defined set of two or more candidate answer values. For example, the clinical criteria can comprise criteria for applicable patients of a medical treatment (e.g., a clinical trial, a medical procedure, a medication, and other types of medical treatments) and the clinical question can ask whether the patient satisfies the criteria. In another example, the clinical criteria can comprise applicable patients of a medical diagnosis and the clinical question can ask whether the patient satisfies the criteria. In another example, the clinical criteria can comprise criteria for classifying a cancer stage of amongst three of more possible cancer stages and the clinical question can ask: “What stage is the patient's cancers?”.

The computer-executable components further comprise an inferencing component that applies the AI model to the different variations of the textual input data and generates inference responses for each of the different variations, and a response assessment component that determines a final inference response to the clinical question based on a combination of the inference responses and generates a confidence score for the final inference response based on a measure of variability between the inference responses. In various implementations, the different variations of the textual input data comprise different variations of at least one of: the textual prompt, the patient data or the clinical criteria data. To this end, the different variations can comprise different patient data variations of the patient data, and wherein the different patient data variations are clinically synonymous in accordance with a defined clinical ontology. The different variations can also comprise different clinical criteria data variations of the clinical criteria data, wherein the different clinical criteria data variations are clinically synonymous in accordance with the defined clinical ontology. To facilitate this end, the computer-executable components further comprise the computer-executable components further comprise a preprocessing component that extracts entities from the patient data and the clinical criteria data using a named entity recognition process, wherein the input data variation component generates the different patient data variations and the clinical data variations using different entity variations of the entities as provided in the defined clinical ontology

In some embodiments, elements described in connection with the disclosed systems can be embodied in different forms such as a computer-implemented method, a computer program product, or another form.

### DESCRIPTION OF THE DRAWINGS

FIG. 1 illustrates a block diagram of an example, non-limiting computing system that facilitates patient to clinical treatment criteria matching with confidence scores, in accordance with one or more embodiments of the disclosed subject matter.

FIG. 2 illustrates a flow diagram of an example method for generating a confidence score for an answer to a clinical question generated by a LLM, in accordance with one or more embodiments of the disclosed subject matter.

FIG. 3 presents a table demonstrating example input data, output data and confidence scores, in accordance with one or more embodiments of the disclosed subject matter.

FIG. 4 illustrates a flow diagram of an example computer-implemented method for generating a confidence score for an answer to a clinical question generated by a LLM, in accordance with one or more embodiments of the disclosed subject matter.

FIG. 5 illustrates an example computer-implemented method for performing LLM based patient to clinical treatment criteria matching with confidence scores, in accordance with one or more embodiments of the disclosed subject matter.

FIG. 6 illustrates an example computer-implemented method for determining recommended clinical treatments applicable for a patient from amongst a plurality of different clinical treatments available, in accordance with one or more embodiments of the disclosed subject matter.

FIG. 7 illustrates an example computer-implemented method for determining recommended clinical diagnoses applicable for a patient from amongst a plurality of different clinical diagnoses, in accordance with one or more embodiments of the disclosed subject matter.

FIG. 8 illustrates a block diagram of an example, non-limiting operating environment in which one or more embodiments described herein can be facilitated.

### DETAILED DESCRIPTION

The following detailed description is merely illustrative and is not intended to limit embodiments and/or application or uses of embodiments. Furthermore, there is no intention to be bound by any expressed or implied information presented in the preceding Background section, Summary section or in the Detailed Description section.

The subject disclosure provides systems, computer-implemented methods, apparatus and/or computer program products that facilitate LLM based patient to clinical treatment criteria matching with confidence scores. A large language model (LLM) is a type of generative artificial intelligence (AI) designed to understand, generate, and manipulate human language. These models are built using advanced machine learning techniques, particularly deep learning, and are trained on vast amounts of text data. LLMs have demonstrated remarkable success in various natural language processing tasks, such as text generation and question answering. For example, LLMs can generate coherent and contextually relevant text based on a given prompt, making them useful for writing essays, articles, stories, and more. LLMs can also answer questions by extracting and synthesizing information from the text they have been trained on. For example, as applied to one usage scenario in the medical domain, an LLM trained vast amounts of clinical text data can analyze patient electronic health records (EHRs) to identify patients who meet specific criteria for particular treatments, trials, or interventions in association with using natural language processing (NLP) to extract relevant information from unstructured text in medical records.

However, a significant concern with LLMs is their proclivity to hallucinate or make inaccurate predications. In this regard, although conventional AI techniques have mechanisms to determine a confidence score for each new inference which they make on unseen data, for generative AI models such as LLMs, there is no present way to do so. This lack of ability to determine a measure of the accuracy of an inference response generated by an LLM hinders their usage for automating clinical reasoning tasks, as these tasks require a high level of confidence in model output accuracy in accordance with medical regulatory bodies.

With this context in mind, the disclosed subject matter is directed to using an LLM to generate an inference response to a clinical question regarding a patient, wherein the textual input data comprises a textual prompt of the clinical question, patient data comprising electronic medical record (EHR) information for the patient and clinical criteria data comprising clinical criteria related to the clinical question. In accordance with the disclosed techniques, the clinical question can comprise any clinical question for which the answer to the clinical question (that is the output response of the LLM) is always one answer included amounts a defined set of two or more possible answers. In other words, the clinical question can comprise any clinical question regarding a patient that has a categorical answer.

For example, in some implementations, the clinical question can comprise whether the patient satisfies clinical criteria of applicable patients for a particular medical treatment, such as a clinical trial, a medical procedure, a medication, or another type of medical treatment. In accordance with this example, the clinical question includes two possible answers, that is, either a first classification that the patient satisfies the clinical criteria or a second classification that the patient does not satisfy the clinical criteria. With this example, the clinical data evaluated by the LLM can include or correspond to the clinical criteria for receiving the medical treatment, such as inclusion and exclusion criteria for a participating in a particular clinical trial, receiving a particular medication, reciting a particular procedure, and so on. In another example, the clinical question can comprise whether the patient satisfies clinical criteria of applicable patients of a particular diagnosis. In accordance with this example, the clinical question again includes two possible answers, that is, either a first classification that the patient satisfies the clinical criteria or a second classification that the patient does not satisfy the clinical criteria. In another example, the clinical question may correspond to a question having more than two possible answers. For instance, the question may correspond to classifying what stage of a defined set of n stages (e.g., wherein n corresponds to an integer greater than 2, such as stage 1, stage 2, stage 3, stage 4, etc.) is a particular patient's cancer. In accordance with this example, the clinical data evaluated by the LLM can include or correspond to the clinical criteria for each cancer stage diagnosis.

To this end, the LLM can comprise a pretrained LLM trained on vast amount of clinical text describing EHR information for various patients and clinical criteria of various medical treatments, diagnoses and/or other types of clinical classifications to which a patient may be matched with or not. The LLM can thus be used to analyze the EHR information for the patient in view of the clinical criteria data and generate an inference response to a clinical question regarding a patient that has a categorical response.

Unlike similar techniques which rely on generative AI, the disclosed techniques provide a mechanism to generate a confidence score for the LLM response to the clinical question. The confidence score provides or corresponds to a measure of confidence in the accuracy of the inference response generated by the LLM. For example, as applied to using an LLM to determine whether a patient satisfies clinical criteria of a particular clinical treatment, regardless of whether the output response is the first classification or the second classification (e.g., the patient is eligible for the clinical treatment or ineligible for the clinical treatment) the disclosed techniques can generate a confidence score for the output response. For instance, the confidence score can include or correspond to a percentage value between 0 and 100, with a score of 100% being the highest confidence and a score of 0% being the lowest confidence.

In various embodiments, the mechanism for determining the confidence score for the LLM output response employs a two-stage approach. In the first stage, an input variation component takes the input text data for the LLM and creates several different variations of it, each having minor syntactic changes, while also ensuring that the semantics or meaning of the input text data is not altered. For example, the different input data variations comprise different variations of at least one of: the textual prompt, the patient data or the clinical criteria data. In association with generating the different input data variations, the input variation component can employ a defined clinical ontology and ensure the different patient data variations and the different clinical criteria data variations are clinically synonymous in accordance with the defined clinical ontology. These different input data variations are then sent to LLM for inferencing.

In the second stage, a response assessment component performs statistical analysis on the LLM responses from the different input data variations. In this regard, the response assessment component can determine a final response to the clinical question and a measure of confidence in the final response based on the aggregated responses and the degree of variability in the aggregated responses. Various strategies can be employed for this assessment, such as a simple majority, a weighted majority, or confidence thresholds.

For example, in some implementations, the final response can correspond to the majority classification (e.g., either the first classification or the second classification) with respect to the input data variations, and the degree of variability in the results with respect to input data variations is used to associate a real number confidence score between 0 and 100 for the final response. For example, let's assume the clinical question has two categorical answers, say answer A or answer B. The response assessment component can look at all the responses to the different prompt variations, which may all be the same (e.g., all A indicating a consensus that answer A is the correct answer, or all B indicating a consensus that answer B is the correct answer) or different (e.g., some A, some B) in which case the majority answer can be considered the correct and final answer. The response assessment component can also determine a confidence measure that corresponds to a percentage of the answers corresponding to the final answer. For instance, the case where all the answers are the same would correspond to the highest confidence score, or a confidence score of 100%. Likewise, a case where half the answers are A and the other half are B would correspond to the lowest confidence score, or a confidence score of 0%.

In other implementations, the input data variations can include or correspond to different variations of an original input data prompt (e.g., using original terms, phrases and syntax), and the final response can correspond to the original response to the original input data prompt, while the degree of variability of the responses to the input data variations with respect to the original response is used to generate the confidence score for the original response.

The confidence score provides transparency into the reliability of the LLM generated response, helping to build trust in the application. For example, as applied to using an LLM to determine whether a patient matches clinical criteria of respective medical treatments of a plurality of different medical treatments, the disclosed techniques can be used to identify those medical treatment for which the patient is applicable. From amongst those which the patient is applicable, the disclosed techniques can further generate a ranked list of medical treatments for which the patient is applicable, ranked based on the corresponding confidence scores generated for each match. Accordingly, doctors and patients can make more informed decisions about their participation in applicable medical treatments when they understand the level of confidence behind each match. In addition, when the output of the AI is used within a larger system with other AI/non-AI components, it is important for each AI component to provide a measure of its confidence on its output, so that the system as a whole can determine the overall reliability of the results.

Although various embodiments of the disclosed subject matter are described with respect to clinical inferencing, it should be appreciated that the disclosed techniques are not limited to clinical inferencing and can be applied for generating confidence scores for LLMs configured to generate categorical answer responses based on extracting and synthesizing information from text they have been trained on. To this end, the text and the question for which the binary responses are based can relate to essentially any domain and is not limited to the medical or clinical domain.

The terms “algorithm” and “model” are used herein interchangeably unless context warrants particular distinction amongst the terms. The terms “artificial intelligence (AI) model” and “machine learning (ML) model” are used herein interchangeably unless context warrants particular distinction amongst the terms. Reference to an AI or ML model herein can include any type of AI or ML model, including (but not limited to): deep learning (DL) models, neural network models, deep neural network models (DNNs), convolutional neural network models (CNNs), generative adversarial neural network models (GANs), transformer models, and the like. An AI or ML model can include supervised learning models, unsupervised learning models, semi-supervised learning models, combinations thereof, and models employing other types of ML learning techniques. An AI or ML model can include a single model or a group of two or more models (e.g., an ensemble model, chained models, or the like).

One or more embodiments are now described with reference to the drawings, wherein like referenced numerals are used to refer to like elements throughout. In the following description, for purposes of explanation, numerous specific details are set forth in order to provide a more thorough understanding of the one or more embodiments. It is evident, however, in various cases, that the one or more embodiments can be practiced without these specific details.

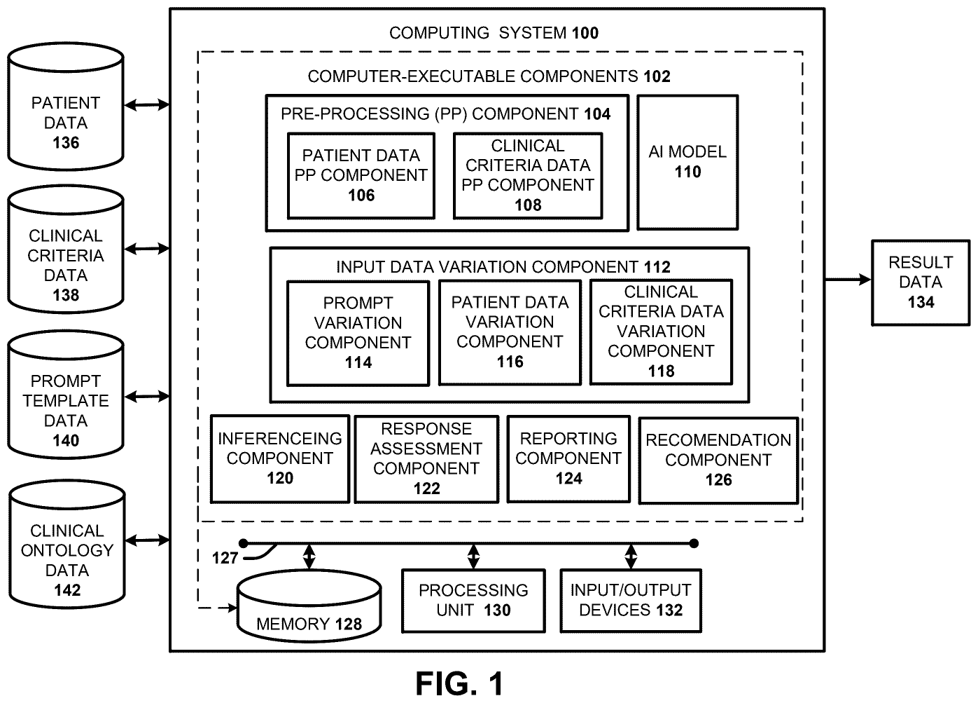

Turning now to the drawings, FIG. 1 illustrates a block diagram of an example, non-limiting computing system 100 that that facilitates LLM based patient to clinical criteria matching with confidence scores, in accordance with one or more embodiments of the disclosed subject matter. Computing system 100 can include or correspond to one or more computing devices, machines, virtual machines, computer-executable components, datastores, and the like that may communicatively coupled to one another either directly or via one or more wired or wireless communication frameworks.

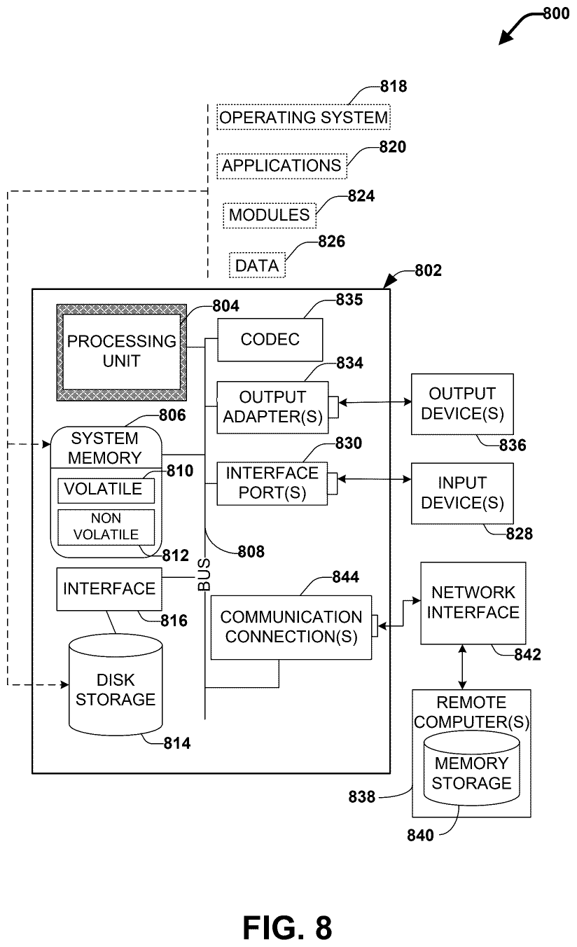

Computing system 100 can include computer-executable (i.e., machine-executable) components or instructions embodied within one or more machines (e.g., embodied in one or more computer-readable storage media associated with one or more machines) that can perform one or more of the operations described with respect to the corresponding components. For example, computing system 100 can include (or be operatively coupled to) at least one memory 128 that stores computer-executable components 102 and at least one processor (e.g., processing unit 130) that executes the computer-executable components 102 stored in the at least one memory 128. These computer-executable components can include, (but are not limited to), pre-processing component 104, AI model 110, input data variation component 112, inferencing component 120, response assessment component 122, reporting component 124 and recommendation component 126. Examples of said memory 128 and processing unit 130 as well as other suitable computer or computing-based elements, can be found with reference to FIG. 8 (e.g., system memory 806 and processing unit 804 respectively), and can be used in connection with implementing one or more the components shown and described in connection with FIG. 1, or other figures disclosed herein.

Computing system 100 can further include one or more input/output devices 132 to facilitate receiving user input and rendering data to users in association with performing various operations described with respect to the machine-executable components 102 and/or processes described herein. Suitable examples of the input/output devices 132 are described with reference to FIG. 8 (e.g., input devices 828 and output devices 836). Computing system 100 can further include a system bus 127 that couples the memory 128, the processing unit 130 and the input/output devices 132 to one another.

In various embodiments, AI model 110 can include or correspond to a generative AI model configured to generate a categorical answer response to a textual prompt question based on analyzing and processing textual input data related to the textual prompt question using NLP techniques. For example, the AI model 110 can include or correspond to a LLM that has been trained on vast amounts of unstructured and/or structured text data corresponding to the textual input data. The textual prompt can include any clinical question regarding a patient for which there is a defined set of two or more categorical answers. For example, in some embodiments, the textual prompt question can include or correspond to a clinical question regarding a patient satisfies clinical criteria. With these embodiments, the AI model 110 can be configured to generate an inference response to the clinical question that comprises either a first classification that the patient satisfies the clinical criteria or a second classification that the patient does not satisfy the clinical criteria, based on comparing relevant textual input patient data for the patient as provided in patient data 136, such as information describing the patients medical history, demographics, and other specific-condition related parameters, with textual clinical criteria input data describing the clinical criteria, as provided in the clinical criteria data 138.

In some embodiments, the clinical criteria can include or correspond to criteria of applicable patients for a particular clinical treatment. For example, the clinical treatment can include or correspond to a clinical trial and the clinical criteria can include inclusion and exclusion criteria of patients for participation in the clinical trial. For instance, the clinical trial criteria can outline the eligibility requirements for participants of the clinical trial, such as age range, disease stage, and previous treatment history. With these embodiments, the clinical criteria data 138 can include or correspond to a database comprising textual descriptions for a plurality of different clinical trials, wherein the textual descriptions describe the inclusion and exclusion criteria for patients applicable/inapplicable to participate in the respective clinical trials. For example, the textual descriptions can include or correspond to unstructured text and/or structured text comprising sentences, paragraphs, and/or the like summarizing the clinical criteria of the respective clinical trials. In another example, the clinical treatment can include or correspond to a medication, a medical device, a medical procedure, a therapy regimen, or another type of medical treatment. With these embodiments, the clinical criteria data 138 can include or correspond to a database comprising textual descriptions of the respective clinical treatments, the textual descriptions describing inclusion and exclusion criteria for patients applicable/inapplicable to receive the respective clinical treatments.

In another embodiment, the clinical criteria can include or correspond to clinical criteria of a particular medical diagnosis. With these embodiments, the AI model 110 can be configured to determine whether a patient satisfies criteria of a particular medical diagnosis or not based on comparing relevant input information for the patient (e.g., medical history information, laboratory reports, radiology reports, clinician notes, etc.) with clinical criteria data (e.g., a textual description) describing inclusion and exclusion criteria for the particular medical diagnosis. To this end, the clinical criteria data 138 can include or correspond to a database comprising textual descriptions of the conditions/parameters of various different possible medical diagnoses.

In another embodiments, the clinical question posted by the text prompt may correspond to a question having more than two possible answers. For instance, the question may correspond to classifying what stage of a defined set of n stages (e.g., wherein n corresponds to an integer greater than 2, such as stage 1, stage 2, stage 3, stage 4, etc.) is a particular patient's cancer. In accordance with this example, the clinical criteria data evaluated by the AI model 110 can include or correspond to the clinical criteria for each cancer stage diagnosis.

To this end, the computing system 100 can be communicatively coupled to various databases or data sources via any suitable wired or wireless communication network. These databases can include but are not limited to, patient data 136, clinical criteria data 138, prompt template data 140 and clinical ontology data 142. The patient data 136 can include or correspond to a database comprising relevant textual patient data for respective patients of a plurality of different patients. For example, the relevant textual patient data for each patient of the plurality of patients can include information describing the patient's medical history, demographics, medications, habits, past and present diagnosis, laboratory data, physiology conditions and so on. The clinical criteria data 138 can include or correspond to a database comprising textual descriptions of the clinical criteria related to the clinical question. For example, as applied to the clinical question being whether a patient is applicable for a particular medical treatment, the clinical criteria data 138 can include textual data (e.g., clinical guideline data or the like) describing the criteria (clinical criteria and non-clinical criteria) for applicable patients for each of a plurality of different medical treatments (e.g., which can correspond to clinical trials, medications, medical procedures, and so on). In another example, as applied to the clinical question regarding whether the patient can be classified with a particular medical diagnosis, the clinical criteria data 138 can describe the inclusion and exclusion criteria for applying various known medical diagnosis, classifying a particular stage of cancer, and so on.

The AI model 110 can further correspond to an LLM trained to determine an answer to the clinical question based on comparing the patient data for the patient to the clinical criteria data using NLP techniques. In this regard, the AI model 110 can include or correspond to a pretrained LLM trained on vast amounts of clinical text including and/or corresponding to the patient data 136 and the clinical criteria data 138. In some embodiments, the AI model 110 can be stored at an external system or device communicatively coupled to the computing system 100 (e.g., as opposed to being stored in memory 128) and accessed by the computing system 100 via any suitable wired or wireless communication network.

In this regard, the textual input data processed by the AI model 110 can include a textual prompt of the clinical question, patient data for a given/selected patient, and clinical criteria data related to the clinical question. The textual prompt controls the task performed by the AI model 110, which in accordance with the disclosed techniques corresponds to a classification task; that is generating an answer corresponding to one answer of amongst a defined set of two or more answer options. For example, in some implementations in which the clinical question comprises whether a patient satisfies clinical criteria of a particular medical treatment or medical diagnosis, the classification task corresponds to a predefined matching task of determining whether a patient matches/satisfies the clinical criteria for a particular medical treatment or diagnosis. For example, in some implementations, the textual prompt fed as input to the AI model 110 can state: “Does the (selected patient) patient satisfy the criteria of the (selected) medical treatment?” or “Does the (selected patient) patient satisfy the criteria of the (selected) medical diagnosis?”. In another example, the clinical question may have more than two defined possible answers, such as a question and that asks, “What stage of cancer does this patient have?”, wherein the type of cancer could be one of n different stages, and wherein n is an integer greater than two.

Thus, in accordance with the disclosed techniques, the textual prompt can be fixed or predefined such that the textual prompt poses a clinical question having a defined set of two or more possible answers corresponding to classifications (e.g., a classification of eligible or ineligible for a particular treatment, a diagnosis classification, or the like). Various different clinical questions and corresponding textual prompts can be predefined and provided in the textual prompt template data 140.

At a high level, the pre-processing component 104 preprocesses the textual input data (e.g., a textual prompt of the clinical question, patient data for a given/selected patient, and clinical criteria data related to the clinical question) using a named entity recognition process to extract relevant terms and phrases (e.g. as facilitated using clinical ontology data 142). The input data variation component 112 further generates different variations of the textual input data by replacing one or more of the terms and phrases with clinically synonymous terms and phrases (and/or values), as facilitated using clinical ontology data 142. The inferencing component 120 further applies the different input variations to the AI model 110 to generate corresponding responses to the clinical question. The response assessment component 122 further evaluates the collective responses and determines a final answer for the clinical question based on the aggregated responses and generates a confidence score for the final answer based on a measure of variability between the responses.

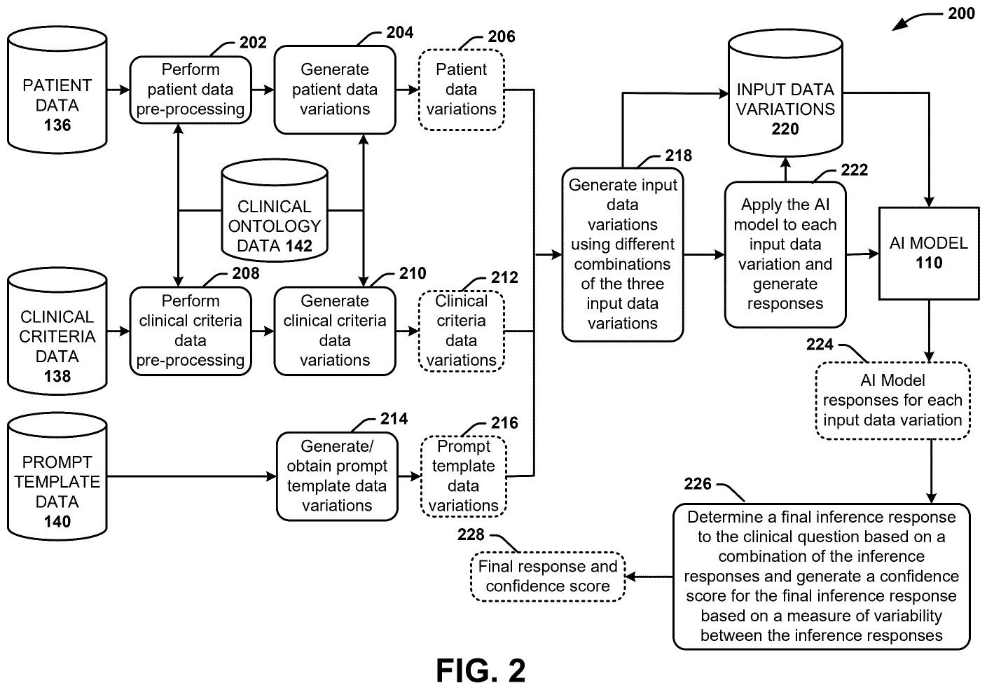

In some embodiments, the reporting component 124 can further generate result data 134 comprising the results of the response assessment component 122. The reporting component 124 can render the result data 134 via any suitable electronic output device (e.g., of input/output devices 132), and/or provide (e.g., via any suitable wired or wireless communication network) the result data 134 to another system, device, or downstream application for additional processing. The recommendation component 126 can also evaluate the results of the response assessment component 122 and generate recommendations regarding recommended clinical treatments and/or diagnoses that are applicable for a given patient based in part on respective confidence scores associated with applicable clinical treatment and/or diagnoses. Additional details regarding the features and functionalities of the pre-processing component 104, the input data variation component 112, the inferencing component 120, the response assessment component 122, the reporting component 124 and the recommendation component 126 are described with below with reference to FIGS. 2-6.

In this regard, FIG. 2 illustrates a flow diagram of an example method 200 for generating a confidence score for an answer to a clinical question generated by an LLM, in accordance with one or more embodiments of the disclosed subject matter. Repetitive description of like elements employed in respective embodiments is omitted for sake of brevity.

With reference to FIG. 2 in view of FIG. 1, method 200 corresponds to an example method for employing the AI model 110 to generate an inference response to a clinical question regarding whether a patient on processing textual input data by the AI model 110, wherein the textual input data comprises a textual prompt of the clinical question (as predefined and provided in prompt template data 140, patient data comprising electronic medical record information for the patient (e.g., as provided in patient data 136) and clinical criteria data comprising the clinical criteria related to the clinical question (e.g., as provided in clinical criteria data 138), and wherein the inference response comprises an answer included amongst a defined set of two or more possible answers. In other words, the clinical question can correspond to any clinical question regarding the patient for which the answer is one value of amongst a possible set of values including two or more values.

In this regard, method 200 is described in accordance with determining whether a selected patient (e.g., of amongst a plurality of different patients represented in the patient data 136) matches the clinical criteria of a particular medical treatment (or diagnosis) as selected from amongst a plurality of different medical treatments (or diagnosis) represented in the clinical criteria data 138. However, method 200 is applicable to a variety of different clinical questions. In addition, it should be appreciated however that method 200 can be repeatedly performed by the computing system 100 for the same patient yet different clinical questions to generate results for each of different clinical questions. For example, the same patient can be evaluated with respect to different clinical trials to determine which of the clinical trials the patient is eligible and/or ineligible for. Likewise, method 200 can be performed for any patient represented in patient data 138.

Thus, given a selected patient and text prompt comprising a clinical question related to the patient, method 200 begins with pre-preprocessing the patient data for the selected patient at 202 (e.g., via patient data pp component 106) and at 208, pre-preprocessing the clinical criteria data related to the clinical question (e.g., via clinical criteria data pp component 108). For example, assuming the clinical question asks whether the patient satisfies clinical criteria for a particular medical treatment, the clinical criteria data 138 would comprise textual guideline data describing the clinical criteria of the particular medical treatment. In another example, assuming the clinical question asks, “What stage is the patient's cancer?”, the clinical criteria data 138 would comprise textual information describing the criteria for each stage classification. To this end, the clinical criteria data 138 is controlled as a function of the text prompts included in the prompt template data 140 and can include or correspond to a database that aggregates clinical guidelines, clinical textbooks, clinical articles, and the like for a variety of different clinical treatments, medications, procedures, diagnoses, and the like.

In general, the pre-processing performed at 202 and 208 corresponds to extracting entities from the patient data and the relevant clinical criteria data using NLP and a named entity recognition process. For example, the patient data 136 can comprise unstructured and/or structured text provided in EHR information for the patient describing the patient's medical history, current and past diagnoses, demographic information, clinician notes, radiology reports, laboratory reports, and so on. In various embodiments, at 202 the patient data pre-processing component 106 can identify relevant parameters of the patient information to the clinical criteria and/or the clinical question and identify these parameters and the corresponding parameter values as included in the patient data 136 for the patient. In some embodiments, at 202 the patient data pre-processing component 202 can further employ the clinical ontology data 142 and a named entity recognition process to identify and extract entities (e.g., defined terms and/or phrases) included in the patient data for the patient that can be replaced/exchanged with clinically synonymous terms.

In this regard, a clinical ontology is a structured framework that organizes information in the healthcare domain to facilitate data sharing, integration, and analysis. It defines a set of concepts and categories in a subject area and shows their properties and the relations between them. For example, the clinical ontology data 142 can define a standardized terminology or common language for describing clinical concepts, such as diseases, symptoms, treatments, and diagnostic procedures. The clinical ontology data 142 can employ hierarchical structure that organizes the clinical concepts into a hierarchy, often with broader categories branching into more specific subcategories, and defines relationships between concepts, such as “is a type of,” “part of,” or “associated with.” The clinical ontology data 142 is further machine readable; that is structured in a way that can be processed by computers, enabling automated reasoning and data analysis. Some examples of clinical ontologies that can be included in the clinical ontology data 142 include but are not limited to: SNOMED CT (Systematized Nomenclature of Medicine—Clinical Terms): A comprehensive clinical terminology that covers diseases, clinical findings, procedures, and other aspects of healthcare; ICD (International Classification of Diseases): A standardized coding system used for diagnosing and classifying diseases and a wide variety of signs, symptoms, abnormal findings, and external causes of injury or diseases; LOINC (Logical Observation Identifiers Names and Codes): A standardized system for identifying medical laboratory observations; and MeSH (Medical Subject Headings): A comprehensive controlled vocabulary for the purpose of indexing journal articles and books in the life sciences.

Named entity recognition (NER) is a subtask of information extraction that seeks to locate and classify named entities mentioned in unstructured text into predefined categories such as the names of persons, organizations, locations, expressions of times, quantities, monetary values, percentages, and more. NER is a fundamental technology in natural language processing (NLP) and is used in various applications such as text analysis, information retrieval, and machine translation. In accordance with the disclosed techniques, at 202 the patient data pp component 106 can employ the clinical ontology data 142 and a NER to identify terms, phrases and/or numerical expressions (e.g., of times, of physiological measurements, and so on) included in the relevant patient data that constitute entities as defined in the clinical ontology and assign a category classification to each of the respective entities in accordance with the defined clinical oncology data 142 (e.g., a procedure, a medication, a location, a symptom, a disease type, a disease stage, etc.).

Similarly, at 208, the clinical criteria data pp component 108 can process the textual clinical criteria data related to the clinical question. For example, assuming the clinical question pertains to a particular medical treatment or diagnosis, the clinical ontology data 142 can define to terms, phrases, and/or numerical expressions corresponding to entities that may be included in the clinical criteria data describing the inclusion and exclusion criteria for the particular medical treatment or diagnosis. Thus, using NER and the clinical ontology data 142, at 202, the clinical criteria pp component 108 can classify the respective entities included in the relevant clinical criteria data as belonging to their respective categories.

At 210, the clinical criteria data variation component 118 can generate a number (n) different clinical criteria data variations of the original clinical criteria (n0) related to the clinical question (e.g., for the particular medical treatment or diagnosis being evaluated or the like). In this regard, the clinical criteria data variations 212 can include the original clinical criteria (n0) related to the clinical question and a number n of different variations of the original clinical criteria n0, wherein the number n can vary. Importantly, the different variations are controlled to be clinically and semantically synonymous. This can be achieved using techniques such as synonym replacement (e.g., using clinically synonymous terms and/or values for one or more of the extracted entities, the clinically synonymous terms and/or values being provided in the clinical ontology data 142), sentence structure modification, and linguistic diversity. For instance, in a simplified example, let's assume the original clinical criteria corresponds to the following textual criteria of a clinical trial:
n0=“The patient must have histologically or cytologically confirmed prostate cancer with plans to start medical castration therapy.”

In accordance with this example, the clinical criteria data variations n generated at 210 might include the following four clinically synonymous variations:
n1=“The patient should have confirmed prostate cancer, either histologically or cytologically, and must be planning to start medical castration therapy.”
n2—“To participate, a patient needs histological or cytological confirmation of prostate cancer and plans for medical castration therapy.”
n3=“Eligibility requires a diagnosis of prostate cancer confirmed histologically or cytologically, with the intention of undergoing medical castration therapy.”
n4=“Participants should have a confirmed diagnosis of prostate cancer through histological or cytological means and be intending to start medical castration therapy.”

In this regard, the generation of the clinical criteria data variations performed at 210 can correspond to a paraphrasing process that involves re-paraphrasing the original clinical criteria in different ways to create meaningful variations thereof, while preserving the core meaning of the original clinical criteria. The number of different clinical criteria variations n can be adjusted based on the complexity of the original clinical criteria and the desired level of confidence granularity.

In some embodiments, at 204, the patient data variation component 116 can similarly generate a number (m) of different patient data variations of the original clinical patient data (m0) for the particular patient being evaluated. In this regard, the patient data variations 206 can include the original patient data (m0) for the particular patient being evaluated and a number m of different variations of the original patient data m0, wherein the number m can vary. This can also involve using techniques such as synonym replacement (e.g., using clinically synonymous terms and/or values for one or more of the extracted entities from the original patient data extracted at 202, the clinically synonymous terms and/or values being provided in the clinical ontology data 142), sentence structure modification, and linguistic diversity.

As noted above, in various embodiments, the textual prompt providing the clinical question that is input to the AI model 110 can be predefined and fixed. For example, the prompt template data 140 can define a number of different predefined text prompts corresponding to different clinical questions. In some embodiments, different variations of the predefined text prompts are not generated. In other embodiments, in addition to generating different patient data variations and different clinical criteria data variations, at 214, the prompt variation component 114 can also generate or obtain a number of different variations p of the original text prompt p0. In this regard, the prompt template data variations 216 can include the original prompt p0 and one or more additional variations p. For example, assuming the predefined prompt template for a selected clinical question states: “Does the patient satisfy the clinical criteria for treatment “xyz” ?” (wherein treatment xyz can correspond to any selected treatment for which clinical criteria data is available in clinical criteria data 138). In accordance with this example, some suitable prompt variations might include: “Does the patient match the clinical criteria for treatment xyz?”, or “Is the patient eligible for the clinical treatment xyz?”. In this regard, the variations of the text prompt for a clinical question can rephrase the question in a different way so long as the possible answer options of amongst the defined possible answer options remains the same (e.g., yes satisfy or no, doesn't satisfy in this example). In some embodiments, the prompt variation component 114 can generate the different prompts which have the same meaning using same or similar techniques described above (e.g., synonym replacement, sentence structure modification, and linguistic variation). In other embodiments, the prompt template data 140 can provide/define the applicable prompt variations p for an original prompt p0.

Continuing with process 200, at 218 the input data variation component 112 can generate input data variations 220 using different combinations of the patient data variations 208, the clinical criteria data variations 212 and/or the prompt template data variations 218. In this regard, the input to the AI model 110 includes three textual components, a textual prompt of the clinical question, let's call component Q, the patient's data, let's call component P, and the clinical criteria data, let's call component C. In various embodiments, the input data variations 220 generated at 218 can include the original formulation of these three components, that is the original question prompt (Q0), the original patient data (P0) and the original clinical criteria data (C0). The input data variations 220 can further include various additional input data variations that vary with respect to at least one of the three components. In this regard, the additional input data variations can include different prompt template variations 216, different clinical criteria data variations 212 and/or different patient data variations 206. For example, different variations may include the same clinical question prompt yet different versions of the patient data and/or the clinical criteria data, such as (Q0, P0, C1), (Q0, P0, C2), (Q0, P0, C3), (Q0, P1, C0), (Q0, P2, C0), (Q0, P1, C1), and so on. The number n of different input data variations 220 generated at 218 can vary depending on the complexity of the clinical criteria and the level of confidence granularity desired. In this regard, the different input data variations can be denoted as I0-In, wherein n can correspond to any integer greater than 1.

At 222, the inferencing component 120 can apply the AI model 110 to each input data variation I0-In and generate corresponding responses or answers, denoted as answers A0-An. In this regard, the AI model responses 224 generated for each input data variation I0, I1, I2 . . . In will be one answer of the defined set of two or more possible answers (e.g., yes or no, eligible not eligible, a particular value of amongst 3 or more possible values, etc.).

At 226, the response assessment component 122 can further determine a final inference response to the clinical question based on a combination of the inferences responses (e.g., answers A0-An) and generate a confidence score for the final inference response based on a measure of variability between the inference responses (e.g., final response and confidence score 228). In other words, at 226, the response assessment component 122 performs statistical analysis on the AI model responses 224 from the different input data variations to determine a final response to the clinical question and a measure of confidence in the final response based on the aggregated responses and the degree of variability in the aggregated responses. Various strategies can be employed for this assessment, such as a simple majority, a weighted majority, or confidence thresholds.

For example, in some implementations, the final response can correspond to the majority response, and determining the confidence score can be based on the number of responses of amongst the different responses that are the majority. Importantly, because the responses are respectively one answer of amongst a defined set of two or more possible answers, the degree of variability can be computed as a real number corresponding to a confidence score between 0 and 100. For instance, let's assume the possible responses to the clinical question include either a first classification that the patient satisfies clinical criteria for a particular medical treatment or a second classification that the patient does not satisfy the clinical criteria. Let's further assume 10 different input data variations (e.g., I1-I10) are used which resulted in all 10 responses (e.g., A1-A10) being the same classification (e.g., either the first classification or the second classification). In accordance with this example, and the simple majority protocol, the final response will be the majority classification (i.e., the same classification) and the confidence score will be 100 (e.g., 10 out 10 the same input variations resulted in the same classification). In another example, let's assume the 10 different input data variations resulted in 7 responses having the first classification and 3 responses having the second classification. In accordance with this example, the final response would be the majority classification (i.e., the first classification) and the confidence score would be 70% (e.g., 7 out of the 10 responses where congruent). In yet another example, let's assume 10 different input variations are used, which resulted in 5 responses having the first classification and 5 responses having the second classification. In this example, because no majority response is observed the response assessment component 122 can determine that the answer to the clinical question cannot be determined accurately and output a final response to that effect. For example, the response assessment component 122 can calculate the confidence score as 0% and indicate the AI model is indeterminate of the answer to the clinical question.

Although the above simple majority examples involve binary answer options, the same techniques can be applied to clinical questions with three or more possible answer options. For instance, let's say the answer options include option A, option B, option C and option D. In association with determining the final response based on simple majority, the final response would correspond to the answer option having the highest number of responses. For example, let's assume 20 different input data variations are used, resulting in the following response distribution: 8 responses for option A, 5 responses for option B, 5 responses for option C, and 2 responses for option D. Here, option A would be the final response, and the confidence score can reflect the percentage of responses out of all the responses corresponding to option A (e.g., 8/20 or 40%). In other embodiments, a weighted majority protocol can be applied and/or confidence thresholds.

In other embodiments, the input data variations 224 can include the original textual input data (e.g., the original prompt, patient data and clinical criteria data, (e.g., Q0, P0 and C0)), and the final response can correspond to the original response to the original input data (e.g., A0), while the degree of variability of the responses to the additional input data variations with respect to the original response can be used by the response assessment component 122 used to generate the confidence score for the original response (e.g., the confidence score is based on the number of responses A1-An corresponding to A0).

In some embodiments method 200 can be used to answer a clinical question that corresponds to whether a selected patient (e.g., of amongst a plurality of different patients represented in the patient data 136) matches the clinical criteria of a particular medical treatment (or diagnosis) as selected from amongst a plurality of different medical treatments (or diagnosis) represented in the clinical criteria data 138. In some implementations of these embodiments, method 200 can be repeatedly performed by the computing system 100 for the same patient yet different clinical treatments (or diagnoses) to generate results for each of the different medical treatments (or diagnoses) represented in the clinical criteria data 138.

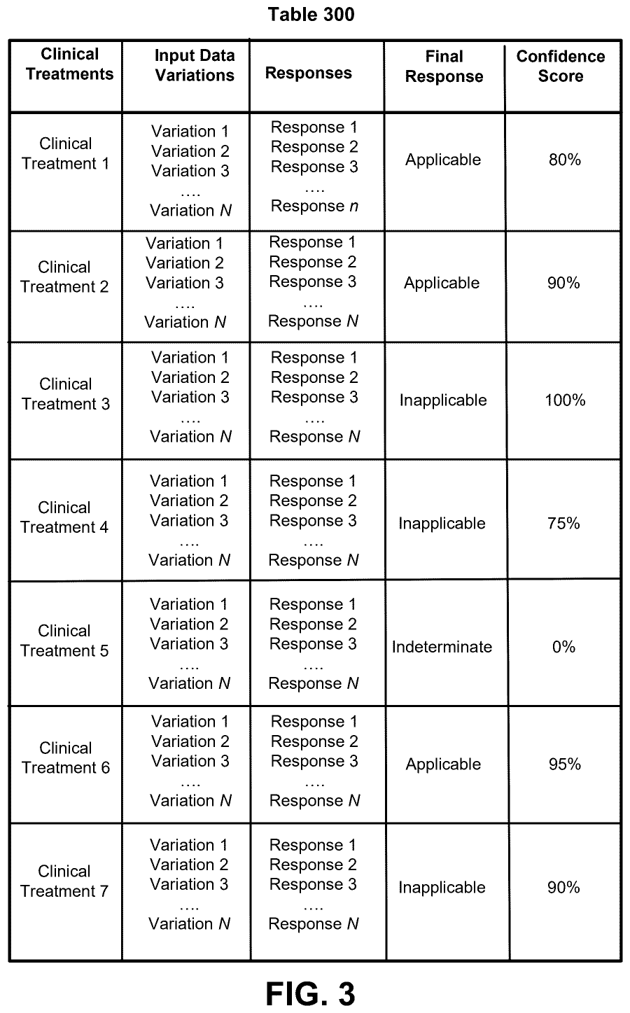

For example, FIG. 3 presents a table 300 summarizing results of process 200 as applied to evaluate a patient's applicability or eligibility for 6 different clinical treatments. It should be appreciated that 6 different clinical treatments are shown for sake of example, and that the number of different clinical treatments evaluated can vary and include any number (e.g., hundreds, thousands, etc.). With reference to FIG. 3 in view of FIGS. 1 and 2, as shown in table 300, the input data variations for each clinical treatment evaluated respectively include 1-N different input data variations. These input data variations for each clinical treatment correspond to the input data variations 220. Each input data variation for a given clinical treatment was further processed by the AI model 110 to generate corresponding responses 1-N (corresponding to AI model responses 224), each response being one of two classifications, either the patient is applicable for the clinical treatment or the patient is inapplicable for the clinical treatment. The final output data of process 200 for each clinical treatment evaluated includes a final response to the clinical question (e.g., as determined in accordance with step 226 of process 200), which is whether the clinical treatment is applicable for the patient, inapplicable for the patient, or indeterminate (e.g., corresponding to a 0% confidence result), and a confidence score determined for each final response based on a measure of variability between the corresponding responses 1-N for the given clinical treatment.

Thus, in some embodiments, process 200 can be performed by the computing system 100 to determine whether a patient is applicable or inapplicable for a particular clinical treatment and/or diagnosis and generate a confidence score representing a measure of accuracy in the AI model's inference response. With these embodiments, the result data 134 shown in FIG. 1 can correspond to a single, final response/answer to the clinical question indicating that the patient is applicable or inapplicable (or indeterminate) and include the confidence score for the single, final response/answer.

In other embodiments, process 200 can be performed by the computing system repeatedly for the same patient yet different clinical treatments/diagnosis to generate result data corresponding to that presented in Table 300. In some implementations of these embodiments, the response assessment component 122 can further evaluate the results for each clinical treatment/diagnosis evaluated to generate a ranked list of clinical treatments for which the patient is applicable, wherein the order of the ranking is based on the corresponding confidence scores. For example, applicable clinical treatments associated with higher confidence scores can be ranked higher than applicable clinical treatments associated with lower confidence scores. The reporting component 124 can further generate result data 134 in the form of a report providing the ranked list of applicable clinical treatments and their corresponding confidence scores. The recommendation component 126 can also generate recommendation data included in the result data 134 recommending one or more of the applicable clinical treatments (or diagnoses) that satisfy a defined recommendation criterion (e.g., having a confidence score above a threshold, recommend the top X number of applicable clinical treatments, or the like).

Still in other embodiments, process 200 can be performed by the computing system 100 repeatedly for the same clinical treatment (or diagnosis) yet different patients to generate result data identifying respective patients for which the clinical treatment (or diagnosis) is applicable and corresponding confidence scores. The reporting component 124 can further generate result data 134 in the form of a report providing a ranked list of applicable patients and their corresponding confidence scores. The recommendation component 126 can also generate recommendation data included in the result data 134 recommending one or more of the applicable patients that satisfy a defined recommendation criterion (e.g., having a confidence score above a threshold, recommend the top Y number of applicable patients, or the like).

In some embodiments, in association with performing process 200 for the same clinical patient yet as applied to different clinical treatments (or different diagnoses), the computing system 100 can perform the generation of the patient data variations 206 only once, and store the different patient data variations (e.g., in memory 128 or another memory device accessible to the computing system 100) for re-use when performing process 200 as applied to different clinical criteria for different clinical treatments. As a result, the processing speed or computation time (and processing power) involved in evaluating whether a patient matches clinical criteria of a plurality of different clinical treatments or diagnoses represented in the clinical criteria data 138 can be significantly reduced. In addition, in some embodiments, the different clinical criteria data variation 212 generated for each clinical treatment criteria (and/or clinical diagnosis criteria) represented in the clinical criteria data 142 can also be generated in an offline manner before runtime (e.g., before processing at 218 to generate the input data variations 220), further reducing the processing speed or computation time involved in matching patients to respective clinical criteria of different clinical treatment and/or diagnoses with confidence scores. Likewise, in other embodiments, the patient data variations 206 (for a single patient and/or a plurality of different patients), the clinical criteria data variations 212 (for a single clinical treatment or diagnosis or a plurality of different ones), and the prompt template data variations 216 can be generated in an offline manner prior to generation of the input data variations 220.

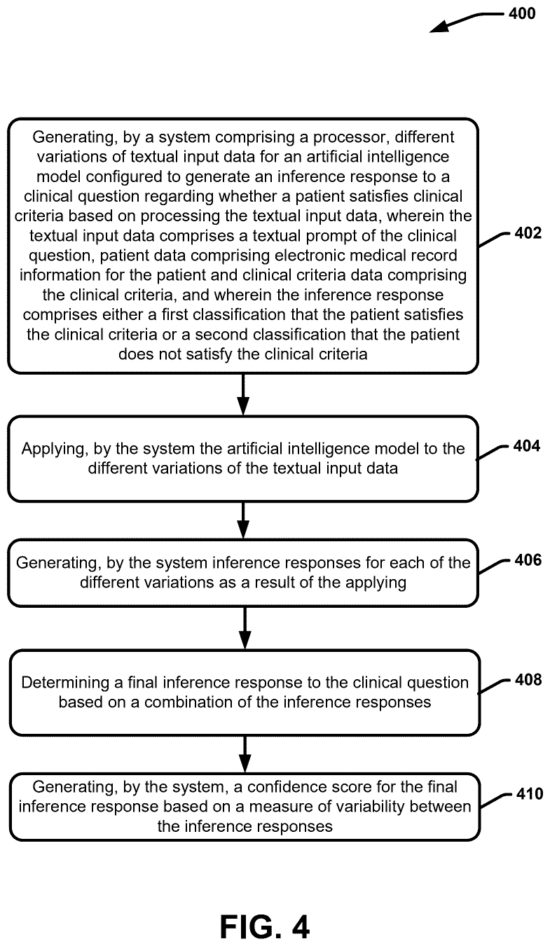

FIG. 4 illustrates a flow diagram of an example, computer-implemented method for generating a confidence score for an answer to a clinical question generated by a LLM, in accordance with one or more embodiments of the disclosed subject matter. With reference to FIG. 4 in view FIGS. 1-3, method 400 comprises, at 402, generating (e.g., via input data variation component 112), by a system comprising a processor (e.g., computing system 100), different variations of textual input data for an AI model (e.g., AI model 110) configured to generate an inference response to a clinical question regarding a patient, wherein the textual input data comprises a textual prompt of the clinical question, patient data comprising electronic medical record information for the patient and clinical criteria data comprising clinical criteria related to the clinical question, and wherein the inference response comprises an answer value of amongst a defined set of two or more candidate answer values (e.g., eligible or not eligible, yes or no, answer A, B or C, etc.). At 404, method 400 comprises applying, by the system (e.g., via inferencing component 120), the AI model to the different variations of the textual input data. At 406, method 400 comprises generating, by the system, inference responses for each of the different variations as a result of the applying (e.g., via the AI model 110). At 408, method 400 comprises determining, by the system (e.g., via response assessment component 122), a final inference response to the clinical question based on a combination of the inference responses. For example, the final inference response can correspond to the majority response, a weighted majority, or the like. At 410, method 400 comprises generating, by the system, a confidence score for the final inference response based on a measure of variability between the inference responses (e.g., via response assessment component 122). For example, in some implementations, the confidence score can correspond to the percentage of responses out of all the responses that are the final inference response. As noted above, this calculation can be accurately and efficiently performed because system 100 controls or restricts the clinical question is to be any clinical question that has a defined set of two or more possible answer values.

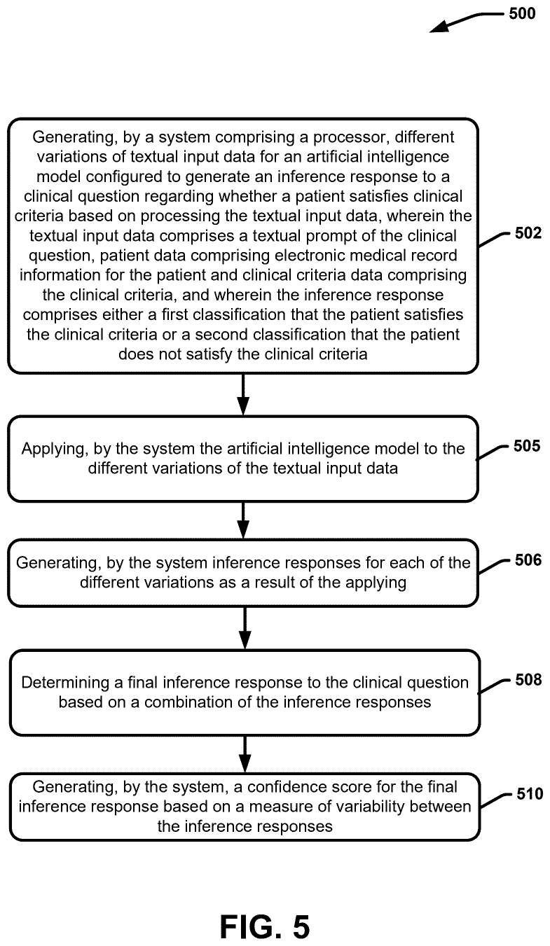

FIG. 5 illustrates a flow diagram of an example, computer-implemented method for generating a confidence score for an answer to a clinical question generated by a LLM, in accordance with one or more embodiments of the disclosed subject matter. With reference to FIG. 5 in view FIGS. 1-3, method 500 comprises, at 502, generating (e.g., via input data variation component 112), by a system comprising a processor (e.g., computing system 100), different variations of textual input data for an AI model (e.g., AI model 110) configured to generate an inference response to a clinical question regarding whether a patient satisfies clinical criteria based on processing the textual input data, wherein the textual input data comprises a textual prompt of the clinical question, patient data comprising electronic medical record information for the patient and clinical criteria data comprising the clinical criteria, and wherein the inference response comprises either a first classification that the patient satisfies the clinical criteria or a second classification that the patient does not satisfy the clinical criteria. At 504, method 500 comprises applying, by the system (e.g., via inferencing component 120), the AI model to the different variations of the textual input data. At 506, method 500 comprises generating, by the system, inference responses for each of the different variations as a result of the applying (e.g., via the AI model 110). At 508, method 500 comprises determining, by the system (e.g., via response assessment component 122), a final inference response to the clinical question based on a combination of the inference responses. For example, the final inference response can correspond to the majority response, a weighted majority, or the like. At 510, method 500 comprises generating, by the system, a confidence score for the final inference response based on a measure of variability between the inference responses (e.g., via response assessment component 122). For example, in some implementations, the confidence score can correspond to the percentage of responses out of all the responses that are the final inference response. As noted above, this calculation can be accurately and efficiently performed because the clinical question is controlled or restricted to be any clinical question that has a defined set of two or more possible answer values.

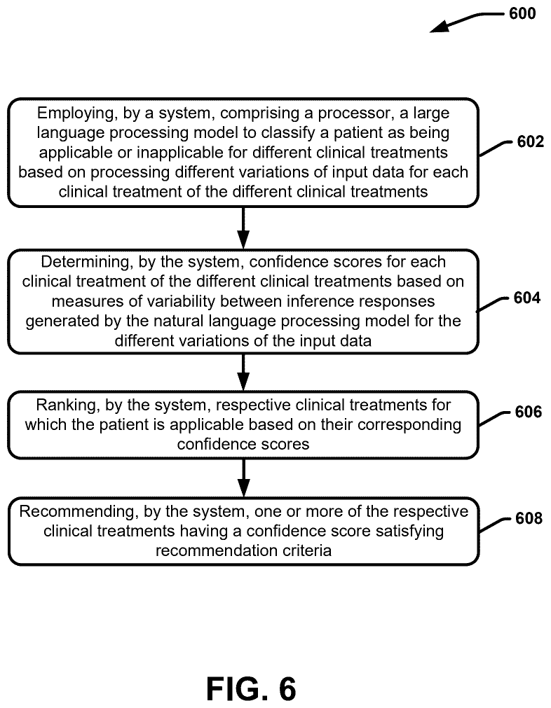

FIG. 6 illustrates an example computer-implemented method 600 for determining recommended clinical treatments applicable for a patient from amongst a plurality of different clinical treatments available, in accordance with one or more embodiments of the disclosed subject matter. With reference to FIG. 6 in view FIGS. 1-3, method 600 comprises, at 602, employing, by a system comprising a processor (e.g., computing system 100), a LLM to classify a patient as being applicable or inapplicable for different clinical treatments based on processing different variations of input data for each clinical treatment of the different clinical treatments. At 604, method 600 comprises determining, by the system, confidence scores for each clinical treatment of the different clinical treatments based on measures of variability between inference responses generated by the LLM for the different variations of the input data. At 606, method 600 comprises ranking, by the system, respective clinical treatments for which the patient is applicable based on their corresponding confidence scores. At 608, method 600 comprises recommending, by the system, one or more of the respective clinical treatments having a confidence score satisfying recommendation criteria.

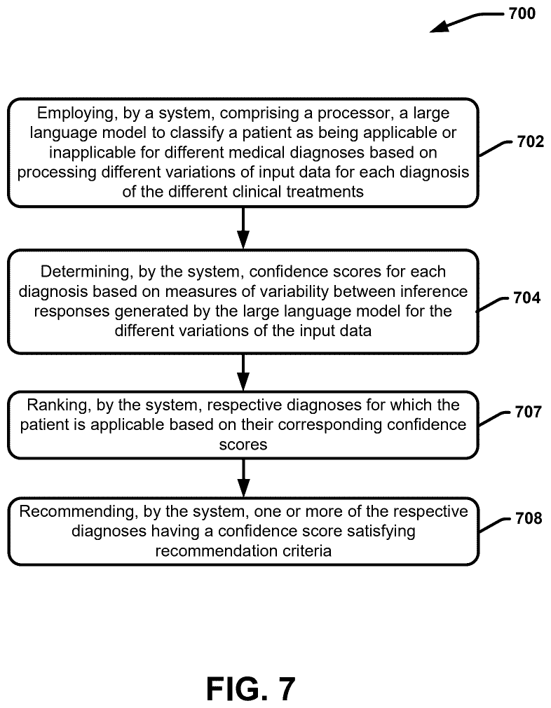

FIG. 7 illustrates an example computer-implemented method 600 for determining recommended clinical diagnoses applicable for a patient from amongst a plurality of different clinical diagnoses, in accordance with one or more embodiments of the disclosed subject matter. With reference to FIG. 7 in view FIGS. 1-3, method 700 comprises, at 702, employing, by a system comprising a processor (e.g., computing system 100), a LLM to classify a patient as being applicable or inapplicable for different clinical diagnoses based on processing different variations of input data for each diagnosis of the different clinical diagnoses. At 704, method 700 comprises determining, by the system, confidence scores for each diagnosis of the different clinical diagnoses based on measures of variability between inference responses generated by the LLM for the different variations of the input data. At 706, method 700 comprises ranking, by the system, respective diagnoses for which the patient is applicable based on their corresponding confidence scores. At 708, method 700 comprises recommending, by the system, one or more of the respective diagnoses having a confidence score satisfying recommendation criteria.

### Example Operating Environments

One or more embodiments can be a system, a method, and/or a computer program product at any possible technical detail level of integration. The computer program product can include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium can be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire. To this end, the a computer readable storage medium, a machine-readable storage medium, or the like as used herein can include a non-transitory computer readable storage medium, a non-transitory machine-readable storage medium, and the like.

Computer readable program instructions described herein can be downloaded to respective computing/processing devices from a computer readable storage medium or to an external computer or external storage device via a network, for example, the Internet, a local area network, a wide area network and/or a wireless network. The network can comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention can be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions can execute entirely on the user's computer, partly on the user's computer, as a stand-alone software package, partly on the user's computer and partly on a remote computer or entirely on the remote computer or server. In the latter scenario, the remote computer can be connected to the user's computer through any type of network, including a local area network (LAN) or a wide area network (WAN), or the connection can be made to an external computer (for example, through the Internet using an Internet Service Provider). In some embodiments, electronic circuitry including, for example, programmable logic circuitry, field-programmable gate arrays (FPGA), or programmable logic arrays (PLA) can execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It can be understood that each block of the flowchart illustrations and/or block diagrams, and combinations of blocks in the flowchart illustrations and/or block diagrams, can be implemented by computer readable program instructions.

These computer readable program instructions can be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions can also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions can also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams can represent a module, segment, or portion of instructions, which comprises one or more executable instructions for implementing the specified logical function(s). In some alternative implementations, the functions noted in the blocks can occur out of the order noted in the Figures. For example, two blocks shown in succession may, in fact, be executed substantially concurrently, or the blocks can sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

In connection with FIG. 8, the systems and processes described below can be embodied within hardware, such as a single integrated circuit (IC) chip, multiple ICs, an application specific integrated circuit (ASIC), or the like. Further, the order in which some or all of the process blocks appear in each process should not be deemed limiting. Rather, it should be understood that some of the process blocks can be executed in a variety of orders, not all of which can be explicitly illustrated herein.

With reference to FIG. 8, an example environment 800 for implementing various aspects of the claimed subject matter includes a computer 802. The computer 802 includes a processing unit 804, a system memory 806, a codec 835, and a system bus 808. The system bus 808 couples system components including, but not limited to, the system memory 806 to the processing unit 804. The processing unit 804 can be any of various available processors. Dual microprocessors and other multiprocessor architectures also can be employed as the processing unit 804.

The system bus 808 can be any of several types of bus structure(s) including the memory bus or memory controller, a peripheral bus or external bus, or a local bus using any variety of available bus architectures including, but not limited to, Industrial Standard Architecture (ISA), Micro-Channel Architecture (MSA), Extended ISA (EISA), Intelligent Drive Electronics (IDE), VESA Local Bus (VLB), Peripheral Component Interconnect (PCI), Card Bus, Universal Serial Bus (USB), Advanced Graphics Port (AGP), Personal Computer Memory Card International Association bus (PCMCIA), Firewire (IEEE 1384), and Small Computer Systems Interface (SCSI).

The system memory 806 includes volatile memory 810 and non-volatile memory 812, which can employ one or more of the disclosed memory architectures, in various embodiments. The basic input/output system (BIOS), containing the basic routines to transfer information between elements within the computer 802, such as during start-up, is stored in non-volatile memory 812. In addition, according to present innovations, codec 835 can include at least one of an encoder or decoder, wherein the at least one of an encoder or decoder can consist of hardware, software, or a combination of hardware and software. Although, codec 835 is depicted as a separate component, codec 835 can be contained within non-volatile memory 812. By way of illustration, and not limitation, non-volatile memory 812 can include read only memory (ROM), programmable ROM (PROM), electrically programmable ROM (EPROM), electrically erasable programmable ROM (EEPROM), Flash memory, 3D Flash memory, or resistive memory such as resistive random access memory (RRAM). Non-volatile memory 812 can employ one or more of the disclosed memory devices, in at least some embodiments. Moreover, non-volatile memory 812 can be computer memory (e.g., physically integrated with computer 802 or a mainboard thereof), or removable memory. Examples of suitable removable memory with which disclosed embodiments can be implemented can include a secure digital (SD) card, a compact Flash (CF) card, a universal serial bus (USB) memory stick, or the like. Volatile memory 810 includes random access memory (RAM), which acts as external cache memory, and can also employ one or more disclosed memory devices in various embodiments. By way of illustration and not limitation, RAM is available in many forms such as static RAM (SRAM), dynamic RAM (DRAM), synchronous DRAM (SDRAM), double data rate SDRAM (DDR SDRAM), and enhanced SDRAM (ESDRAM) and so forth.

Computer 802 can also include removable/non-removable, volatile/non-volatile computer storage medium. FIG. 8 illustrates, for example, disk storage 814. Disk storage 814 includes, but is not limited to, devices like a magnetic disk drive, solid state disk (SSD), flash memory card, or memory stick. In addition, disk storage 814 can include storage medium separately or in combination with other storage medium including, but not limited to, an optical disk drive such as a compact disk ROM device (CD-ROM), CD recordable drive (CD-R Drive), CD rewritable drive (CD-RW Drive) or a digital versatile disk ROM drive (DVD-ROM). To facilitate connection of the disk storage 814 to the system bus 808, a removable or non-removable interface is typically used, such as interface 816. It is appreciated that disk storage 814 can store information related to a user. Such information might be stored at or provided to a server or to an application running on a user device. In one embodiment, the user can be notified (e.g., by way of output device(s) 836) of the types of information that are stored to disk storage 814 or transmitted to the server or application. The user can be provided the opportunity to opt-in or opt-out of having such information collected or shared with the server or application (e.g., by way of input from input device(s) 828).

It is to be appreciated that FIG. 8 describes software that acts as an intermediary between users and the basic computer resources described in the suitable operating environment 800. Such software includes an operating system 810. Operating system 810, which can be stored on disk storage 814, acts to control and allocate resources of the computer 802. Applications 820 take advantage of the management of resources by operating system 810 through program modules 824, and program data 826, such as the boot/shutdown transaction table and the like, stored either in system memory 806 or on disk storage 814. It is to be appreciated that the claimed subject matter can be implemented with various operating systems or combinations of operating systems.

A user enters commands or information into the computer 802 through input device(s) 828. Input devices 828 include, but are not limited to, a pointing device such as a mouse, trackball, stylus, touch pad, keyboard, microphone, joystick, game pad, satellite dish, scanner, TV tuner card, digital camera, digital video camera, web camera, and the like. These and other input devices connect to the processing unit 804 through the system bus 808 via interface port(s) 830. Interface port(s) 830 include, for example, a serial port, a parallel port, a game port, and a universal serial bus (USB). Output device(s) 836 use some of the same type of ports as input device(s) 828. Thus, for example, a USB port can be used to provide input to computer 802 and to output information from computer 802 to an output device 836. Output adapter 834 is provided to illustrate that there are some output devices 836 like monitors, speakers, and printers, among other output devices 836, which require special adapters. The output adapters 834 include, by way of illustration and not limitation, video and sound cards that provide a means of connection between the output device 836 and the system bus 808. It should be noted that other devices or systems of devices provide both input and output capabilities such as remote computer(s) 838.

Computer 802 can operate in a networked environment using logical connections to one or more remote computers, such as remote computer(s) 838. The remote computer(s) 838 can be a personal computer, a server, a router, a network PC, a workstation, a microprocessor based appliance, a peer device, a smart phone, a tablet, or other network node, and typically includes many of the elements described relative to computer 802. For purposes of brevity, only a memory storage device 840 is illustrated with remote computer(s) 838. Remote computer(s) 838 is logically connected to computer 802 through a network interface 842 and then connected via communication connection(s) 844. Network interface 842 encompasses wire or wireless communication networks such as local-area networks (LAN) and wide-area networks (WAN) and cellular networks. LAN technologies include Fiber Distributed Data Interface (FDDI), Copper Distributed Data Interface (CDDI), Ethernet, Token Ring and the like. WAN technologies include, but are not limited to, point-to-point links, circuit switching networks like Integrated Services Digital Networks (ISDN) and variations thereon, packet switching networks, and Digital Subscriber Lines (DSL).

Communication connection(s) 844 refers to the hardware/software employed to connect the network interface 842 to the bus 808. While communication connection 844 is shown for illustrative clarity inside computer 802, it can also be external to computer 802. The hardware/software necessary for connection to the network interface 842 includes, for exemplary purposes only, internal and external technologies such as, modems including regular telephone grade modems, cable modems and DSL modems, ISDN adapters, and wired and wireless Ethernet cards, hubs, and routers.

It is to be noted that aspects or features of this disclosure can be exploited in substantially any wireless telecommunication or radio technology, e.g., Wi-Fi; Bluetooth; Worldwide Interoperability for Microwave Access (WiMAX); Enhanced General Packet Radio Service (Enhanced GPRS); Third Generation Partnership Project (3GPP) Long Term Evolution (LTE); Third Generation Partnership Project 2 (3GPP2) Ultra Mobile Broadband (UMB); 3GPP Universal Mobile Telecommunication System (UMTS); High Speed Packet Access (HSPA); High Speed Downlink Packet Access (HSDPA); High Speed Uplink Packet Access (HSUPA); GSM (Global System for Mobile Communications) EDGE (Enhanced Data Rates for GSM Evolution) Radio Access Network (GERAN); UMTS Terrestrial Radio Access Network (UTRAN); LTE Advanced (LTE-A); etc. Additionally, some or all of the aspects described herein can be exploited in legacy telecommunication technologies, e.g., GSM. In addition, mobile as well non-mobile networks (e.g., the Internet, data service network such as internet protocol television (IPTV), etc.) can exploit aspects or features described herein.

While the subject matter has been described above in the general context of computer-executable instructions of a computer program that runs on a computer and/or computers, those skilled in the art will recognize that this disclosure also can or may be implemented in combination with other program modules. Generally, program modules include routines, programs, components, data structures, etc. that perform particular tasks and/or implement particular abstract data types. Moreover, those skilled in the art will appreciate that the inventive methods may be practiced with other computer system configurations, including single-processor or multiprocessor computer systems, mini-computing devices, mainframe computers, as well as personal computers, hand-held computing devices (e.g., PDA, phone), microprocessor-based or programmable consumer or industrial electronics, and the like. The illustrated aspects may also be practiced in distributed computing environments where tasks are performed by remote processing devices that are linked through a communications network. However, some, if not all aspects of this disclosure can be practiced on stand-alone computers. In a distributed computing environment, program modules may be located in both local and remote memory storage devices.

As used in this application, the terms “component,” “system,” “platform,” “interface,” and the like, can refer to and/or can include a computer-related entity or an entity related to an operational machine with one or more specific functionalities. The entities disclosed herein can be either hardware, a combination of hardware and software, software, or software in execution. For example, a component may be, but is not limited to being, a process running on a processor, a processor, an object, an executable, a thread of execution, a program, and/or a computer. By way of illustration, both an application running on a server and the server can be a component. One or more components may reside within a process and/or thread of execution and a component may be localized on one computer and/or distributed between two or more computers.

In another example, respective components can execute from various computer readable media having various data structures stored thereon. The components may communicate via local and/or remote processes such as in accordance with a signal having one or more data packets (e.g., data from one component interacting with another component in a local system, distributed system, and/or across a network such as the Internet with other systems via the signal). As another example, a component can be an apparatus with specific functionality provided by mechanical parts operated by electric or electronic circuitry, which is operated by a software or firmware application executed by a processor. In such a case, the processor can be internal or external to the apparatus and can execute at least a part of the software or firmware application. As yet another example, a component can be an apparatus that provides specific functionality through electronic components without mechanical parts, wherein the electronic components can include a processor or other means to execute software or firmware that confers at least in part the functionality of the electronic components. In an aspect, a component can emulate an electronic component via a virtual machine, e.g., within a cloud computing system.

In addition, the term “or” is intended to mean an inclusive “or” rather than an exclusive “or.” That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. Moreover, articles “a” and “an” as used in the subject specification and annexed drawings should generally be construed to mean “one or more” unless specified otherwise or clear from context to be directed to a singular form.

As used herein, the terms “example” and/or “exemplary” are utilized to mean serving as an example, instance, or illustration. For the avoidance of doubt, the subject matter disclosed herein is not limited by such examples. In addition, any aspect or design described herein as an “example” and/or “exemplary” is not necessarily to be construed as preferred or advantageous over other aspects or designs, nor is it meant to preclude equivalent exemplary structures and techniques known to those of ordinary skill in the art.

Various aspects or features described herein can be implemented as a method, apparatus, system, or article of manufacture using standard programming or engineering techniques. In addition, various aspects or features disclosed in this disclosure can be realized through program modules that implement at least one or more of the methods disclosed herein, the program modules being stored in a memory and executed by at least a processor. Other combinations of hardware and software or hardware and firmware can enable or implement aspects described herein, including a disclosed method(s). The term “article of manufacture” as used herein can encompass a computer program accessible from any computer-readable device, carrier, or storage media. For example, computer readable storage media can include but are not limited to magnetic storage devices (e.g., hard disk, floppy disk, magnetic strips . . . ), optical discs (e.g., compact disc (CD), digital versatile disc (DVD), blu-ray disc (BD) . . . ), smart cards, and flash memory devices (e.g., card, stick, key drive . . . ), or the like.

As it is employed in the subject specification, the term “processor” can refer to substantially any computing processing unit or device comprising, but not limited to, single-core processors; single-processors with software multithread execution capability; multi-core processors; multi-core processors with software multithread execution capability; multi-core processors with hardware multithread technology; parallel platforms; and parallel platforms with distributed shared memory. Additionally, a processor can refer to an integrated circuit, an application specific integrated circuit (ASIC), a digital signal processor (DSP), a field programmable gate array (FPGA), a programmable logic controller (PLC), a complex programmable logic device (CPLD), a discrete gate or transistor logic, discrete hardware components, or any combination thereof designed to perform the functions described herein. Further, processors can exploit nano-scale architectures such as, but not limited to, molecular and quantum-dot based transistors, switches and gates, in order to optimize space usage or enhance performance of user equipment. A processor may also be implemented as a combination of computing processing units.

In this disclosure, terms such as “store,” “storage,” “data store,” data storage,” “database,” and substantially any other information storage component relevant to operation and functionality of a component are utilized to refer to “memory components,” entities embodied in a “memory,” or components comprising a memory. It is to be appreciated that memory and/or memory components described herein can be either volatile memory or nonvolatile memory, or can include both volatile and nonvolatile memory.

By way of illustration, and not limitation, nonvolatile memory can include read only memory (ROM), programmable ROM (PROM), electrically programmable ROM (EPROM), electrically erasable ROM (EEPROM), flash memory, or nonvolatile random access memory (RAM) (e.g., ferroelectric RAM (FeRAM). Volatile memory can include RAM, which can act as external cache memory, for example. By way of illustration and not limitation, RAM is available in many forms such as synchronous RAM (SRAM), dynamic RAM (DRAM), synchronous DRAM (SDRAM), double data rate SDRAM (DDR SDRAM), enhanced SDRAM (ESDRAM), Synchlink DRAM (SLDRAM), direct Rambus RAM (DRRAM), direct Rambus dynamic RAM (DRDRAM), and Rambus dynamic RAM (RDRAM). Additionally, the disclosed memory components of systems or methods herein are intended to include, without being limited to including, these and any other suitable types of memory.

It is to be appreciated and understood that components, as described with regard to a particular system or method, can include the same or similar functionality as respective components (e.g., respectively named components or similarly named components) as described with regard to other systems or methods disclosed herein.

What has been described above includes examples of systems and methods that provide advantages of this disclosure. It is, of course, not possible to describe every conceivable combination of components or methods for purposes of describing this disclosure, but one of ordinary skill in the art may recognize that many further combinations and permutations of this disclosure are possible. Furthermore, to the extent that the terms “includes,” “has,” “possesses,” and the like are used in the detailed description, claims, appendices and drawings such terms are intended to be inclusive in a manner similar to the term “comprising” as “comprising” is interpreted when employed as a transitional word in a claim.

What is claimed is:

1. A system, comprising:
at least one memory that stores computer-executable components; and
at least one processor that executes the computer-executable components stored in the at least one memory, wherein the computer-executable components comprise:
an input variation component that generates different variations of textual input data for an artificial intelligence model configured to generate an inference response to a clinical question regarding a patient, wherein the textual input data comprises a textual prompt of the clinical question, patient data comprising electronic medical record information for the patient and clinical criteria data comprising clinical criteria related to the clinical question, and wherein the inference response comprises an answer value of amongst a defined set of two or more candidate answer values;
an inferencing component that applies the artificial intelligence model to the different variations of the textual input data and generates inference responses for each of the different variations; and
a response assessment component that determines a final inference response to the clinical question based on a combination of the inference responses and generates a confidence score for the final inference response based on a measure of variability between the inference responses.

2. The system of claim 1, wherein the artificial intelligence model comprises a large language model.

3. The system of claim 1, wherein the different variations comprise different variations of at least one of: the textual prompt, the patient data or the clinical criteria data.

4. The system of claim 1, wherein the different variations comprise different prompt variations of the textual prompt.

5. The system of claim 1, wherein the different variations comprise different patient data variations of the patient data, and wherein the different patient data variations are clinically synonymous in accordance with a defined clinical ontology.

6. The system of claim 5, wherein the computer-executable components further comprise:
a preprocessing component that extracts entities from the patient data using a named entity recognition process; and
a patient data variation component that generates the different patient data variations using different entity variations of the entities as provided in the defined clinical ontology.

7. The system of claim 1, wherein the different variations comprise different clinical criteria data variations of the clinical criteria data, and wherein the different clinical criteria data variations are clinically synonymous in accordance with a defined clinical ontology.

8. The system of claim 7, wherein the computer-executable components further comprise:
a preprocessing component that extracts entities from the clinical criteria data using a named entity recognition process; and
a clinical criteria data variation component that generates the different clinical criteria data variations using different entity variations of the entities as provided in the defined clinical ontology.

9. The system of claim 1, wherein the clinical criteria comprises criteria of patients for receiving a particular medical treatment or diagnosis, and wherein the clinical question asks whether the patient satisfies the clinical criteria.

10. The system of claim 9, wherein the computer-executable components further comprise:
a recommendation component that generates recommendation data recommending application of the medical treatment or the medical diagnosis for the patient based on the confidence score exceeding a threshold confidence score.

11. A method, comprising:
generating, by a system comprising a processor, different variations of textual input data for an artificial intelligence model configured to generate an inference response to a clinical question regarding a patient, wherein the textual input data comprises a textual prompt of the clinical question, patient data comprising electronic medical record information for the patient and clinical criteria data comprising clinical criteria related to the clinical question, and wherein the inference response comprises an answer value of amongst a defined set of two or more candidate answer values;
applying, by the system, the artificial intelligence model to the different variations of the textual input data;
generating, by the system, inference responses for each of the different variations as a result of the applying;
determining, by the system, a final inference response to the clinical question based on a combination of the inference responses; and
generating, by the system, a confidence score for the final inference response based on a measure of variability between the inference responses.

12. The method of claim 11, wherein the artificial intelligence model comprises a large language model.

13. The method of claim 11, wherein the different variations comprise different variations of at least one of: the textual prompt, the patient data or the clinical criteria data.

14. The method of claim 11, wherein the different variations comprise different patient data variations of the patient data, and wherein the different patient data variations are clinically synonymous in accordance with a defined clinical ontology.

15. The method of claim 14, further comprising:
extracting, by the system, entities from the patient data using a named entity recognition process; and
generating, by the system, the different patient data variations using different entity variations of the entities as provided in the defined clinical ontology.

16. The method of claim 11, wherein the different variations comprise different clinical criteria data variations of the clinical criteria data, and wherein the different clinical criteria data variations are clinically synonymous in accordance with a defined clinical ontology.

17. The method of claim 16, further comprising:
extracting, by the system, entities from the clinical criteria data using a named entity recognition process; and
generating, by the system, the different clinical criteria data variations using different variations of the entities as provided in the defined clinical ontology.

18. The method of claim 11, wherein the clinical criteria comprises criteria of patients for receiving a particular medical treatment or diagnosis, and wherein the clinical question asks whether the patient satisfies the clinical criteria.

19. A non-transitory machine-readable storage medium, comprising executable instructions that, when executed by a processor, facilitate performance of operations, comprising:
generating different variations of textual input data for an artificial intelligence model configured to generate an inference response to a clinical question regarding a patient, wherein the textual input data comprises a textual prompt of the clinical question, patient data comprising electronic medical record information for the patient and clinical criteria data comprising clinical criteria related to the clinical question, and wherein the inference response comprises an answer value of amongst a defined set of two or more candidate answer values;
applying the artificial intelligence model to the different variations of the textual input data;
generating inference responses for each of the different variations as a result of the applying;
determining a final inference response to the clinical question based on a combination of the inference responses; and
generating a confidence score for the final inference response based on a measure of variability between the inference responses.

20. The non-transitory machine-readable storage medium of claim 19, wherein the different variations comprise different clinical criteria data variations of the clinical criteria data, and wherein the operations further comprise:
extracting entities from the clinical criteria data using a named entity recognition process; and
generating the different clinical criteria data variations using different variations of the entities as provided in a defined clinical ontology.
